(19) United States
(12) Patent Application Publication
(10) Pub. No.: US 2021/0030830 A1
(43) Pub. Date: Feb. 4, 2021

(54) COMPOSITION FOR IMPROVING QUANTITY OF TEAR FLUID, COMPOSITION FOR IMPROVING CONSTIPATION, AND COMPOSITION FOR IMPROVING SKIN QUALITY

(71) Applicant: Tokiwa Phytochemical Co., Ltd., Sakura-shi (JP)

(72) Inventors: Jinwei YANG, Sakura-shi (JP); Tomoko KUNIYOSHI, Sakura-shi (JP)

(21) Appl. No.: 16/727,411

(22) Filed: Dec. 26, 2019

(30) Foreign Application Priority Data
Jul. 31, 2019 (JP) 2019-141840

(51) Int. Cl. A61K 36/906; A61K 8/9794; A61P 27/04; A61P 1/10; A61Q 19/08; A61K 9/00

(52) U.S. Cl. CPC A61K 36/906; A61K 8/9794; A61K 9/0056; A61P 1/10; A61Q 19/08; A61P 27/04

(57) ABSTRACT

The present invention relates to a composition for improving the quantity of tear fluid, a composition for improving constipation, and a composition for improving skin quality. By containing the extract of Black ginger (Kaempferia parviflora)), it is possible that the quantity of tear fluid, constipation, and skin quality are improved.

### TECHNICAL FIELD

The present invention is related to a composition for improving the quantity of tear fluid, a composition for improving constipation, and a composition for improving skin quality.

### RELATED ART

Black ginger (Kaempferia parviflora) is a plant in the genus Kaempferia of the plant family Zingiberaceae, which is known as Thai ginseng or Kra chai dahm.

Black ginger is cultivated widely in Thai where it is the place of origin, and it is taken on a daily basis from old times. It is used for nourishing tonic, increasing energy, lowering a blood-sugar level, physical recovery, and recovering circulatory organs.

In recent years, many effects of Black ginger (Kaempferia parviflora) are reported such as fat decomposition-promoting action (NON-PATENT DOCUMENT 1, NON-PATENT DOCUMENT 2, PATENT DOCUMENT 1), anti-inflammatory action (NON-PATENT DOCUMENT 3), activation effect for a longevity gene (NON-PATENT DOCUMENT 4, PATENT DOCUMENT 2), anti-saccharification action (NON-PATENT DOCUMENT 4, NON-PATENT DOCUMENT 5), antiallergic action (NON-PATENT DOCUMENT 6), xanthine oxidase inhibition action (PATENT DOCUMENT 3), AMPK activation effect (PATENT DOCUMENT 4), production-inhibiting activity for TNF-a and IL-6 (PATENT DOCUMENT 5), energy metabolism promoting action in muscle cells (PATENT DOCUMENT 6), reinforcement effect for muscle amount and endurance of muscle (PATENT DOCUMENT 7, PATENT DOCUMENT 8, PATENT DOCUMENT 9), cerebral function improvement action (PATENT DOCUMENT 10), and improving action for cold-sensitive constitution (PATENT DOCUMENT 11).

Further, in the human study, reducing effect for abdominal fat and neutral fat in the blood (NON-PATENT DOCUMENT 7), promoting effect for fat combustion in exercise (NON-PATENT DOCUMENT 8), and reinforcement effect for muscle strength and motion function (NON-PATENT DOCUMENT 9, NON-PATENT DOCUMENT 10, NON-PATENT DOCUMENT 11) are confirmed. The application for the human is expected.

### REFERENCE FOR RELATED ART

### Non-Patent Documents

NON-PATENT DOCUMENT 1 Antiobesity effects of Kaempferia parviflor in spontaneously obese type II diabetic mice. J Nat Med, 65, 73-80 (2011).
NON-PATENT DOCUMENT 2 Preventive effect of Kaempferia parviflor ethyl acetate extract and its major components polymethoxyflavonoid on metabolic diseases, Fitoterapia, 82, 1272-1278 (2011).
NON-PATENT DOCUMENT 3 Suppressive effects of methoxyflavonoids isolated from Kaempferia parviflor on inducible nitric oxide synthase (iNOS) expression in RAW 274.7 cells. Journal of Ethnopharmacology, 136(3), 488-495 (2011).
NON-PATENT DOCUMENT 4 Potent SIRT1 Enzyme-stimulating and Anti-glycation Activities of Polymethoxyflavonoids from Kaempferia parviflor, Natural Product Communications, 9(9), 1291-1294 (2014).
NON-PATENT DOCUMENT 5 Antiglycative effect of Kaempferia parviflor Wall. Ex. Baker (Zingiberaceae): Prevention of advanced glycation end product formation, Glycative Stress Research, 5 (4): 163-170 (2018).
NON-PATENT DOCUMENT 6 Evaluation of the biological activity of Black ginger (Kaempferia parviflora) by using in vitro assay, Journal of Nutritional Food, 12(2), 29-35 (2009).
NON-PATENT DOCUMENT 7 Daily intake of Kaempferia parviflor extract decreases abdominal fat in overweight and preobese subjects: a randomized, double-blind, placebo-controlled clinical study, Diabetes, Metabolic Syndrome and Obesity, 11, 447-458(2018).
NON-PATENT DOCUMENT 8 Effects of single oral intake of Kaempferia parviflor extract on energy metabolism—A randomized double-blind crossover study, Jpn Pharmacol Ther, 44(12), 1757-1762 (2016).
NON-PATENT DOCUMENT 9 Enhancement of physical fitness by black ginger extract rich in polymethoxyflavones: a double-blind randomized crossover trial, Integr Mol Med., 3(2), 628-634 (2016).
NON-PATENT DOCUMENT 10 Positive modulation effect of 8-week consumption of Kaempferia parviflor on health-related physical fitness and oxidative status in healthy elderly volunteers, Evid Based Complement Alternat Med., Article ID 732816, doi:10.1155/2012/732816 (2012).
NON-PATENT DOCUMENT 11 Effects of Kaempferia parviflor extract on physical function in elderly subjects—A randomized, double-blind, placebo-controlled parallel-group study—, Jpn Pharmacol Ther, 47(6), 927-36 (2019).

### Patent Documents

PATENT DOCUMENT 1 Japanese Patent Application Publication No. 2013-153397
PATENT DOCUMENT 2 Japanese Patent No. 6417630
PATENT DOCUMENT 3 Japanese Patent No. 5756264
PATENT DOCUMENT 4 Japanese Patent Application Publication No. 2017-31121
PATENT DOCUMENT 5 Japanese Patent Application Publication No. 2017-031120
PATENT DOCUMENT 6 WO2016/163245
PATENT DOCUMENT 7 Japanese Patent Application Publication No. 2017-078032
PATENT DOCUMENT 8 Japanese Patent Application Publication No. 2015-10078
PATENT DOCUMENT 9 Japanese Patent Application Publication No. 2016-008180
PATENT DOCUMENT 10 Japanese Patent Application Publication No. 2018-177654
PATENT DOCUMENT 11 Japanese Patent Application Publication No. 2009-67731

### DESCRIPTION OF THE INVENTION

### Solution to the Problems

However, the effect of Black ginger is not fully verified, and the other effects other than the above-described effects are also expected.

Therefore, concerning the above-described problems, it is an object of the present invention to provide compositions of Black ginger (Kaempferia parviflora), which show new effects.

### Means for Solving the Problems

As a result of investigating the above-described problems, the inventors found that the extract of Black ginger shows improvement for the quantity of tear fluids, constipation, and skin quality in a human study, and complete the present invention.

Namely, one aspect of the present invention is the composition for improving the quantity of tear fluid, which includes the extract of Black ginger (Kaempferia parviflora) as an active ingredient.

More, another aspect of the present invention is the composition for improving constipation, which includes the extract of Black ginger (Kaempferia parviflora) as an active ingredient.

More, another aspect of the present invention is the composition for improving skin quality, which includes the extract of Black ginger (Kaempferia parviflora) as an active ingredient.

### Effects of the Invention

Therefore, the present invention can provide a composition for improving constipation, constipation, or skin quality.

Therefore, concerning the above-described problems, it is an object of the present invention to provide compositions of Black ginger (Kaempferia parviflora), which show new effects.

### BEST MODE FOR CARRYING OUT THE PRESENT INVENTION

Hereinafter, the embodiment of the present invention is described with reference to the drawings. However, the present invention can be carried out with various embodiments and is not limited to the embodiment described below.

This embodiment of the present invention is a composition for improving the quantity of tear fluid, constipation, and skin quality. Concretely, it is the composition for improving the quantity of tear fluid, constipation, and skin quality, which contains an extract as an active ingredient, which is extracted from Black ginger (Kaempferia parviflora).

It is desirable that the part of the material of the Black ginger (Kaempferia parviflora) contains rhizome, however, it is not limited to it as long as it shows the above-identified effects.

More, it is desirable that the place of origin of Black ginger is Southeast Asian Nations such as Thailand and Laos, and Okinawa of Japan, however, it is not limited to them.

More, the dried or raw rhizome of Black ginger can be used as a material and subterranean parts of Black ginger can also be used.

The method of extracting the extract from Black ginger is not limited and it is possible to adopt a well-known method. It is desirable that the extractant is water, hot water, an alcohol solvent, or other organic solvents such as acetone. When the extractant is an alcohol solvent, it is desirable that it is methanol, ethanol, propanol, isopropanol, butanol, iso-butanol, and a mixture of them. When the extractant is an organic solvent, it is desirable that it is an ester such as ethyl acetate, a polyhydric alcohol such as ethylene glycol, propylene glycol or 1,3-butylene glycol, or an ether such as diethyl ether. The solvents can be used singly or combinedly. More, it is desirable that the extractant is a water-containing hydrophilic solvent that contains water and hydrophilic organic solvent. When the extractant is water-containing hydrophilic solvent, the blend ratio of the solvent can be adjusted appropriately. Further, it is desirable that the extractant is ethanol or water-containing ethanol which contains water not less than 30 vol % and not greater than 95 vol %.

The quantity of the extractant is not limited as long as it is possible to obtain the composition that shows the above-described effect. However, it is desirable that the quantity of the extract is not less than 2 parts by weight and not greater than 100 parts by weight when the part by weight of rhizome is assumed 1.

More, it is desirable that the temperature of extraction is not less than 4-degree centigrade and not greater than 90-degree centigrade and the time of extraction is not less than 30 minutes and not greater than 1 week.

The extraction can be performed by any arbitrary method such as stirring extraction, immersion extraction, counter-current extraction, ultrasonic extraction, or supercritical extraction.

The form of the extract of the Black ginger can be a filtrate itself which is obtained by filtering the extracted liquid, a concentrate which is obtained by concentrating the filtrate, a dry product which is obtained by drying the concentrate, a partial purified product of them, a purification product of them, or a mixture of at least one of them.

The method of concentration can be carried out by various methods such as evaporative concentration or membrane concentration.

The method of drying can be carried out by various methods such as drying under reduced pressure, freeze-drying or spray drying.

Further, it is possible to add diluting agent such as a dextrin if necessary.

Furthermore, the method of purification can be carried out by using one or more well-known methods in person skilled in the art such as synthetic adsorption resin, active carbon, ion exchange resin, Sephadex, gel filter media such as bio gel, column chromatography or recrystallization.

More, it is desirable that the form of the composition of the present invention is a food composition, a pharmaceutical composition or a cosmetic composition. Further, when the form is food composition, it is desirable that the form of the composition is such as functional food or health food. Furthermore, in the case, it is possible to take not only the form of solid but also the form of a drink.

More, when the form of the composition is food composition, it is desirable that the form is a drink, a candy, a jelly, or a gummy. When the form of the composition is health food or functional food, it is desirable that the form is a tablet, a hard capsule, a soft capsule, a powder, or a drink.

More, when the form of the composition is a pharmaceutical composition, it is desirable that the form is suitable for oral administration such as a tablet, a capsule, a pill, a liquid, or an emulsion. More, it is possible that the composition includes carriers such as an excipient, a coloring agent, a sweetness enhancing agent, and a suspending agent within the range of pharmaceutically allowable.

More, when the form of the composition is a cosmetic composition, it is desirable that the form of the composition is a lotion, a cream, a pack, a gel, an emulsion, a balsam, an ointment, a liquid, a powder, or other forms which can be applied to local part or internal part of the body.

More, it is desirable that the quantity of the extraction of Black ginger of the composition is adjustable appropriately according to prospected intake.

More, for obtaining the effect of improving the quantity of tear fluid, constipation, or skin quality by the composition, it is desirable that the intake of the composition is not less than 10 mg and not greater than 500 mg per one day, and more desirable that it is not less than 20 mg and not greater than 300 mg per one day, further desirable that it is not less than 50 mg and not greater than 150 mg per one day.

More, the composition of Black ginger contains at least five polymethoxyflavonoids (3,5,7,3′,4′-Pentamethoxyflavone, 5,7,4′-Trimethoxyflavone, 5,7-Dimethoxyflavone, 3,5,7-Trimethoxyflavone, and 3,5,7,4′-Tetramethoxyflavone). It is desirable that the amount of the five polymethoxyflavonoids is contained not less than 4 wt %, and more desirable that it is contained not less than 10 wt % and not greater than 50 wt %. In the range, it is possible to obtain the effect of the composition.

As described above, the composition shows the effects of improving the quantity of tear fluid, constipation, and skin quantity. Further, the range of the amount of intake is the same as the above-described range, which will be clear in the below-described examples.

Therefore, according to the present invention, it is possible to provide the composition which has effects of improving the quantity of tear fluid, constipation, and skin quality.

### Example

Here, the compositions described in the above-described embodiment were produced actually and the effect of them was confirmed. Hereafter, the result will be described concretely.

(1) The Producing Method of the Composition of Black Ginger (Kaempferia parviflora)

The dried chip of the rhizome of Black ginger (Kaempferia parviflora) was pulverized by a mixer, 80 vol % ethanol was added to the pulverized material so that the amount of ethanol was 1 liter per 100 g of the pulverized material. After that, heating refluxing was performed for 2 hours, and the extraction (first extraction) was obtained after filtration. More, 80% ethanol 0.8 liters was added to the residue, and heating refluxing was performed for 1 hour, and the extraction (second extraction) was obtained after filtration. Further, the first extraction and the second extraction were mixed, vacuum concentration was performed at 50 degree centigrade, an excipient was added, decompression-drying was performed at 60 degree centigrade for one evening, a solid 26.6 g was obtained.

By the measurement of liquid chromatography about the above-described solid, it was confirmed that the extract from Black ginger contained five polymethoxyflavonoids (3,5,7,3′,4′-Pentamethoxyflavone, 5,7,4′-Trimethoxyflavone, 5,7-Dimethoxyflavone, 3,5,7-Trimethoxyflavone, and 3,5,7,4′-Tetramethoxyflavone), and the total consistency of them was not less than 10 wt % and not greater than 20 wt %.

(2) Clinical Test

In this test, tablets that contained 50 mg of the extract of Black ginger (Kaempferia parviflora) per one drop were used as test foods. On the other hand, to be the same as the appearance of the test foods, tablets that contained food coloring instead of the extract of Black ginger, were used as placebo.

A random placebo-controlled double-blind study was carried out. In the study, the examinees were healthy Japanese men and women. The examinees of intervention group took 100 mg (2 tablets per 1 day) of the extract of Black ginger (Kaempferia parviflora) after each breakfast with water or tepid water for 8 weeks. On the other hand, the examinees of the placebo group took placebo tablets with an equal number to the extract tablets.

Schirmer's test in which the quantity of tear fluid is measured after 8 weeks after taking the extracts or placebos and questionnaire survey which relates to beauty were carried out.

(3) The Result of the Clinical Test: Schirmer's Test

TABLE 1

| Value of change between before | Placebo | Intervention |
|---|---|---|
| and after taking the extract | group | group |
| Right eye | −3.5 | 1.3 |
| Left eye | −2.7 | 4.0 |
| Average of right eye and | −3.1 | 2.6 |
| left eye | | |

After 8 weeks of taking the test foods, in the intervention group, the length of the wet part of the test paper increased 4.00 mm at the left eye, 1.3 mm at the right eye, and 2.6 mm on average at both eyes. On the other hand, after 8 weeks of taking the placebo foods, in the placebo group, the length of the wet part of the test paper decreased 2.7 mm at the left eye, 3.5 mm at the right eye, and 3.1 mm on average at both eyes. According to the result shown in the above-described Table 1, it is confirmed that the extract of Black ginger is effective for improving the quantity of tear fluid and dry-eye compared with the placebo.

(4) The Result of the Clinical Test: A Questionnaire Survey

The questionnaire survey was carried out according to the survey items and method for evaluation as described below.

Survey items: “I am constipated”, “wrinkles are conspicuous”, “spots are conspicuous”, “skin dullness (Yellowish, Brownish) are noticeable”, “Applying makeup is not easy”

Method for evaluation: the score was obtained by evaluating the above items by 6 steps.

1. “Never applicable”, 2. “Hardly applicable”, 3. “Not very applicable”, 4. “A little applicable”, 5. “Quite a lot applicable”, 6. “Strongly applicable”

TABLE 2

| Value of score change between before | Placebo | Intervention |
|---|---|---|
| and after taking the extract | group | group |
| I am constipated | 0.4 | −0.4 |
| Wrinkles are conspicuous | 1.0 | 0.3 |
| Spots are conspicuous | 0.6 | −0.2 |
| Skin dullness (Yellowish, | 1.1 | −0.3 |
| Brownish) are noticeable | | |
| Applying makeup is not easy” | 0.8 | −0.3 |

According to the above-described Table 2, compared with the placebo group, it was confirmed that the skin quality and constipation were improved by taking the extract of Black ginger.

Therefore, by the above-described human study, it was confirmed that the composition of the present invention had effects for improving the quantity of tear fluid, constipation, and skin quality.

1. A composition for improving the quantity of tear fluid comprising an extract of Black ginger (Kaempferia parviflora).

2. A composition for improving constipation comprising an extract of Black ginger (Kaempferia parviflora).

3. A composition for improving skin quality comprising an extract of Black ginger (Kaempferia parviflora).

4. The composition according to claim 1, which the composition is a food composition.
